(19) United States
(12) Patent Application Publication
(10) Pub. No.: US 2015/0038592 A1
(43) Pub. Date: Feb. 5, 2015

(54) STABLE WATER-IN-OIL EMULSIONS WITH SPREADABLE OILS

(71) Applicant: BEIERSDORF AG, Hamburg (DE)

(72) Inventors: Stephanie Von Der Fecht, Wedel (DE); Svenja Lena Moellgaard, Hamburg (DE); Petra Koch, Hamburg (DE); Isabel Balcke, Hamburg (DE)

(21) Appl. No.: 14/376,977

(22) PCT Filed: Feb. 12, 2013
(86) PCT No.: PCT/EP2013/052741

§ 371 Date: Aug. 6, 2014

(30) Foreign Application Priority Data
Feb. 16, 2012 (DE) 10 2012 002 951.9

(51) Int. Cl. A61K 8/06; A61K 8/37; A61Q 19/00; A61K 8/34; A61K 8/73; A61K 8/25; A61K 8/39; A61K 8/31

(52) U.S. Cl. CPC A61K 8/064; A61K 8/39; A61K 8/37; A61K 8/31; A61K 8/345; A61K 8/732; A61K 8/25; A61Q 19/00
USPC 514/738; 514/785; 514/770

(57) ABSTRACT

The invention comprises a cosmetic and/or dermatological preparation based on water-in-oil emulsions, comprising at least two W/O emulsifiers and at least one oil having a spreadability value of more than 600 mm²/10 min.

The invention comprises cosmetic and/or dermatological preparation based on a water-in-oil emulsion comprising at least two W/O emulsifiers and at least one oil with a spreadability value greater than 600 mm²/10 min.

As cosmetic or medicinal preparations often emulsions, in particular W/O, O/W, O/W/O or W/O/W emulsions, are used. In general emulsions are understood as meaning heterogeneous systems which consist of two liquids which are immiscible or have only limited miscibility with one another and are usually referred to as phases. In an emulsion, one of the two liquids (W/O) is dispersed in the form of very fine droplets in the other liquid. The liquids (pure or as solutions) are present in an emulsion in a more or less fine distribution, which is generally of only limited stability.

If the two liquids are water and oil and oil droplets are present in finely distributed form in water, then this is an oil-in-water emulsion (O/W emulsion, e.g. milk). The basic character, for example electrical conductivity, sensory properties, ability of the continuous phase to be stained, of an O/W emulsion is determined by the water. In the case of a water-in-oil emulsion (W/O emulsion, e.g. butter), the principle is reversed, with the basic character here being determined by the oil.

The prior art recognizes several essential factors which have a positive influence on the stability and rheology of emulsions.

For their formation and stabilization, emulsions generally require one or more emulsifiers, thickeners and/or consistency regulators in order to be stable over a cosmetically acceptable period, generally 1 year after opening a cosmetic preparation.

A particular challenge is the formulation of flowable emulsions. On account of their pleasant spreadability they are highly valued by the consumer. However, the stable formulation is a technological challenge.

To stabilize emulsions, ethoxylated emulsifiers are often used which, as is generally known, lead to resilient, stable emulsion preparations and can often cover a relatively wide sensory range. However, it is known that ethoxylated emulsifiers act as penetration enhancers, due to their PEG units.

For example, EP 1 192 935 A2 discloses W/O emulsions comprising polyethers such as PEG-45 (dodecyl glycol) copolymer and PEG-22 (dodecyl glycol) copolymer.

The use of polyethylene glycols and/or polyethylene glycol derivatives and modifications thereof are a topic of controversial discussion amongst the public since they are suspected, following topical application, of making the skin more permeable to foreign bodies such as e.g. harmful substances.

Furthermore, under the action of solar radiation, the photo-unstable polyethylene glycol-containing (PEG) emulsifiers can be decomposed and trigger unsightly skin reactions.

For the reasons specified, cosmetic formulations are increasingly being sought by the consumer which are free from this class of substance.

Also, in order to be able to develop W/O emulsions that are sensorially pleasant, in most cases PEG emulsifiers are currently used. Since many consumers are looking for products which are free from this class of substance, it is a technological challenge to stabilize W/O emulsions without these substances and at the same time to achieve a pleasant skin feel.

As regards the stability, the challenge is in particular the scaling-up and, in the case of skin feel, parameters such as “rapid and easy absorption, low stickiness” are difficult to achieve without PEG stabilizers.

It is therefore desirable to provide emulsion preparations without ethoxylated emulsifiers which nevertheless can be varied as widely as possible and are above all else stable emulsions.

Furthermore, cosmetic or dermatological preparations have to satisfy a number of aesthetic and sensory aspects in order to achieve adequate consumer acceptance.

However, it is known that the addition of skin moisturizers often leads to the preparations having a sticky effect.

For W/O emulsions it is known that the viscosity is dependent on the amount of emulsifiers used. If a high concentration is used, creams with a high consistency are obtained; upon reducing the use concentration, although the systems are flowable, they are often no longer stable, and this is evident in particular in up-scaling. Consequently, the formulation of flowable W/O emulsions currently represents a major technological challenge.

The viscosity is a measure of the thickness of a fluid. The inverse value of the viscosity is the fluidity, a measure of the flowability of a fluid. The greater the viscosity, the thicker (less flowable) the fluid; the lower the viscosity, the thinner (more flowable) it is. Viscosity and flowability values in this connection are often stated at room temperature (20° C.).

WO 2009080657 A2 discloses W/O emulsions comprising, besides hydrophobically modified polysaccharides, starches and/or agar, polyglyceryl-4 diisostearate polyhydroxystearate sebacate (Isolan GPS®).

FR 2927535 discloses stable water in oil emulsions as cosmetic preparations comprising esters of fatty acids and polyols. A preferred ester that is given is, amongst others, polyglycerol-4 diisostearate polyhydroxystearate sebacate (Isolan GPS®). Glycerol is preferably selected in the described preparations. The preparations should not be sticky on the skin.

WO 2008/055692 A2 describes silicone-free skin protectants comprising, besides oils and polyols, inter alia as emulsifiers polyglycerol-4 diisostearate polyhydroxystearate sebacate and/or polyglyceryl-2-dipolyhydroxystearate.

DE 60 2004 013 358 T2 discloses multiphase emulsions. The described emulsions are “multiple water-in-oil-in-water emulsions”, i.e. W/O/W systems. Moreover, only polyglyceryl-3 diisostearate is mentioned as W/O emulsifier.

DE 10 2008 028 822 A1 discloses an extensive reference work on emulsifiers and oils. Specifically, cosmetic stick compositions in the form of oil-in-water dispersions/emulsions are described. Stick compositions cannot be referred to as flowable at room temperature.

DE 199 24 277 A1 describes W/O emulsions and their known advantages and disadvantages. The disadvantages are said to be overcome by using interface-active substances from the group of alkylmethicone copolyols.

A property of cosmetic products that is very important to the consumer but at the same time can only be measured quantitatively with difficulty is its texture and sensory feel. The term “texture” is understood as meaning those properties of a cosmetic which are attributed to the structural constitution of the preparation, perceived by sense of touch and contact and can possibly be expressed in mechanical or rheological flow properties. The texture can in particular be tested by means of sensorics. The texture of cosmetic products, which may if appropriate be influenced with the help of additives, is of almost equal importance to the consumer as their objectively establishable effects.

The term “sensorics” is the term used to refer to the scientific discipline which deals with evaluating cosmetic preparations on the basis of sensory impressions. The sensory assessment of a cosmetic is made by reference to the visual, olfactory and haptic impressions.
Visual impressions: all features perceptible by eye (color, shape, structure).
Olfactory impressions: all odor impressions perceptible upon drawing in air through the nose, which can often be differentiated into initial odor (top note), main odor (middle note, body) and after-odor (finale). The volatile substances only released upon use also contribute to the olfactory impression.
Haptic impressions: all sensations of the sense of touch, which relate primarily to structure and consistency of the product.

The sensory analysis makes use of the possibility of integrally including the sensory overall impression of a product. Disadvantages of sensory analysis are the subjectivity of the impression, the ability of the test persons to be easily influenced and the considerable scattering of the results as a consequence. These weaknesses are nowadays countered by using groups of trained test persons, mutual shielding of the testers as well as statistical evaluation of the mostly numerous analytical data.

It was therefore a further object of the present invention to provide preparations which, besides the criteria customary for cosmetics such as compatibility, storage stability and the like, also offer the consumer essential, hitherto unknown cosmetic, in particular sensory, benefits. In particular, it was an aim to provide a storage-stable formulation which imparts care without at the same time being slippery, oily or greasy and at the same time displaying a definite “lotion appearance”, i.e. flowable, and NOT A cream appearance.

The invention is a cosmetic and/or dermatological flowable preparation based on a water-in-oil emulsion comprising at least two W/O emulsifiers and at least one oil with a spreadability value greater than 600 mm²/10 min.

The flowability of the preparation means that the preparation is flowable at room temperature (20° C.).

Preferably, the two W/O emulsifiers differ in their HLB value by at most 1. Preference is given to the combination of emulsifiers with an only small HLB difference of e.g. 0.5, or even the same HLB value. Particular preference is given to the use of exclusively two emulsifiers which are subject in particular to the listed limitations as regards their HLB value.

Preferred W/O emulsifiers chosen are diisostearoyl polyglyceryl-3 dimer dilinoleate and polyglyceryl-4 diisostearate/polyhydroxystearate/sebacates.

Diisostearoyl polyglyceryl-3 dimer dilinoleates are PEG-free emulsifiers and known as Isolan® PDI and polyglyceryl-4 diisostearate/polyhydroxystearate/sebacates are known as Isolan® GPS from Evonik with an HLB value of about 5.

Surprisingly, by using two W/O emulsifiers according to the invention in combination with the readily spreadable oils, it has been possible to solve the stated technical challenges.

The use of a combination of two W/O emulsifiers in combination with one or more readily spreading oils leads to flowable, readily distributable lotions with a pleasant, i.e. non-oily, sensory property.

Preferably, only two W/O emulsifiers according to the invention are used; the fraction of further emulsifiers is then 0% by weight.

The two emulsifiers used according to the invention should preferably be W/O emulsifiers with the same HLB value. Particular preference is given here to the combination of diisostearoyl polyglyceryl-3 dimer dilinoleate and polyglyceryl-4 diisostearate/polyhydroxystearate/sebacate. By using relatively large amounts of readily spreading oils, the sensorics of the preparation according to the invention is surprisingly optimized. Readily spreading oils in this connection are oils with a spreadability value of greater than 600 (qmm/10 min) and preferably a viscosity of less than 10 mPas. Particular preference is given in this connection to isopropyl palmitate.

In a comparative experiment, the viscosities of two W/O emulsion preparations were investigated, as indicated in the table below.

| | Viscosity | |
|---|---|---|
| | in mPas | Appearance and sensorics |
| Example formulation 4 | 5400 | Flowable lotion with pleasant |
| | | skin feel, caring but not oily |
| | | or greasy |
| Example formulation 4 | 26 750 | Rich cream, greasy and oily |
| without isopropyl palmitate, | | |
| instead with 9.5% triisostearin | | |

Spreadability of isopropyl palmitate: 625 (mm²/10 min)
Spreadability of triisostearin: 310 (mm²/10 min)

The W/O emulsion preparation without oils according to the invention exhibited a viscosity of 26 750 mPas and was a rich cream. Only by exchanging an oil with a low spreadability value (triisostearin) for an oil with a high spreadability value (isopropyl palmitate) could the viscosity of the preparation be reduced to 5400 mPas, meaning that a lotion with a pleasant skin feel was obtained as product. It is surprising that by merely replacing an oil was the viscosity of the preparation fundamentally influenced so much.

One or more oils with a spreadability value greater than 600 mm²/10 min should therefore surprisingly be used to change, in particular reduce, the viscosity of the cosmetic water-in-oil emulsions containing them. By exchanging the oil phase for oils with a spreadability value greater than 600 mm²/10 min it is possible to provide preparations which can be individually adapted to consumer wishes.

It was the aim of the invention to provide a storage-stable formulation which imparts care without at the same time being slippery, oily or greasy. This is achieved precisely with the formulation according to the invention. It has more structure and imparts this also by virtue of the sensory impression. The consumer tends to translate this into “imparts care, without displaying unpleasant side effects such as oiliness or greasiness”.

A particular feature is that the formulation is nevertheless flowable, i.e. can be used as lotion. Of lotion appearance, i.e. flowable, in this connection are advantageously formulations which do not have a viscosity greater than 4000 mPas (20° C.) and the sensory “cream” effect.

In this connection, it is also surprising that this does not occur at the expense of storage stability. The person skilled in the art of cosmetics now has the option of satisfying individual consumer wishes for improved sensorics without having to worry about neglecting safety and stability.

Preferably, the preparations according to the invention comprise no further additional emulsifiers. The fraction of additional emulsifiers should thus preferably be below 0.01% by weight, based on the total mass of the preparation, in order to be regarded as in accordance with the invention—without additional emulsifier.

In the same way, according to the invention it is possible to dispense with the addition of polyethylene glycols and/or polyethylene glycol derivatives. The fraction of PEGs is therefore below 1% by weight, in particular 0% by weight, based on the total mass of the preparation.

The oil fraction of the preparations according to the invention is advantageously in the range from 10 to 30% by weight, preferably between 15 and 25% by weight, particularly preferably in the range from 18 to 22 by weight, based on the total mass of the preparation.

According to the invention, the lipid phase here comprises at least one oil with a spreadability value greater than 600 (qmm/10 min). It is preferred if additionally a second oil with a spreadability value greater than 600 (qmm/10 min), preferably greater than 800 mm²/10 min, particularly preferably greater than 1000 mm²/10 min is present.

Preferably, one or more of the oils listed in Table 1 below with a spreadability value greater than 600 mm²/10 min and a viscosity of less than 10 mPas is to be selected.

TABLE 1
Oils with a spreadability value greater than 600 mm²/10 min

| | | Spreadability |
|---|---|---|
| | Viscosity | (20 μl/red band |
| INCI | [mPas] | filter) [mm²/10 min] |
| Isopropyl Palmitate | 6.2 | 625 |
| Isopropyl Myristate | 4.6 | 707 |
| Coco-Caprylate/Caprate | 5 | 755 |
| Isopropyl Isostearate | 9.5 | 790 |
| Cyclomethicone | 4-5 | 804-845 |
| Butyl Glycol | 9.6 | 813 |
| Dicaprylate/Dicaprate | | |
| Dicaprylyl Carbonate | 6.3 | 875 |
| Isopropyl Stearate | 6.8 | 910 |
| Ethylhexyl Cocoate | 7.9 | 930 |
| Dibutyl Adipate | 5 | 935 |
| Isodecyl Neopentanoate | 3.8 | 962 |
| Isohexadecane | 3.7 | 990 |
| C13-16 Isoparaffin | 2.6 | 1018 |
| Dicaprylyl Ether | 3.3 | 1020 |

Preferred oils to be used are isopropyl palmitate with a spreadability value of about 625 mm²/10 min and C13-16 isoparaffin with a spreadability value of about 1018 mm²/10 min.

Based on the total preparation, the fraction of oils with a spreadability value greater than 600 mm²/10 min is advantageously 1 to 30, preferably 5 to 25, particularly preferably 10 to 20% by weight, based on the total mass of the preparation. Based on the total mass of oil phase, the fraction of oils with a spreadability value greater than 600 mm²/10 min is advantageously 30 to 90% by weight, preferably 40 to 80, particularly preferably 40 to 60% by weight, based on the total mass of the oil phase.

Surprisingly, by using the readily spreadable oils it has been possible to formulate stable, flowable (at RT) W/O emulsions.

Preference is given to preparations based on a water-in-oil emulsion comprising an oil with a spreadability value greater than 600 mm²/10 min and an oil with a spreadability value above 800 mm²/10 min; it is particularly advantageous if the second spreadable oil has a spreadability value above 1000 mm²/10 min.

In this connection, oils with a spreadability value greater than 600 qmm/10 min and a viscosity less than 10 mPas were advantageous, especially advantageous with a spreadability value between 1020 and 600, namely isopropyl palmitate and/or C13-16 isoparaffin.

At 25° C., isopropyl palmitate has a viscosity of 6.2 mPas, measured with the help of a Viscotester VT-02, from Haake, determined at 25° C. with the help of rotating body 1 or 2 and reading off scale 1 or 2, respectively, corresponding to a shear rate of 10 Pa/s. The ability to spread out to a greater or lesser extent on the skin is referred to in cosmetics as spreadability. The higher the spreadability of an oil, the easier it spreads on the human skin.

The spreadability value (in mm²/10 min) is advantageously measured according to the following procedure: 20 μl of the substance to be investigated are dripped into the middle of a Rotband filter paper from Schleicher & Schüll. A stopwatch is started immediately and after 10 minutes the area is measured which has been wetted by the substance within this time. The measurement is carried out in a temperature-constant room at 25° C.+/−1° C.

Hydrophilic stabilizers such as thickeners and fatty alcohols can optionally be present according to the invention.

The preparations according to the invention advantageously comprise at least one skin moisturizer in a fraction of in total 5 to 20% by weight, based on the total mass of the preparation. Preference is given to a glycerol content of 7 to 15% by weight, particularly preferably of 10% by weight.

By using the emulsifier combination according to the invention, it has been possible to formulate a W/O emulsion which, despite large amounts of skin moisturizers such as glycerol, has a very pleasant skin feel and is stable even in the case of stress tests such as e.g. storage at elevated and/or fluctuating temperatures. This stability exists not only for the 1 to 5 kg batches produced on the laboratory scale, but also for larger amounts such as e.g. 100 to 500 kg and even during production on the ton-scale. This process, known as “up-scaling”, is known to be particularly sensitive for customary W/O emulsions. For the preparations according to the invention, the up-scaling process has proven to be surprisingly problem-free.

Emulsifiers make it possible for two immiscible liquids (for example oil in water) to be able to combine to give an emulsion. On account of the amphiphilic character, they penetrate with their fat-soluble part into the oil. By virtue of the hydrophilic part, the oil droplet now produced by stirring can be dispersed in the aqueous environment. Emulsifiers have primarily no detersive, surfactant character. Emulsifiers reduce the interfacial tension between the two phases and, besides reducing the interfacial work, also achieve a stabilization of the emulsion formed. They stabilize the formed emulsion by means of interfacial films, as well as by forming steric or electrical barriers, as a result of which the merging (coalescence) of the emulsified particles is prevented.

In order that compounds can be effective as emulsifiers, they must have a certain molecular structure. A structural characteristic of such compounds is their amphiphilic molecular structure. The molecule of such a compound has at least one group with affinity to substances of strong polarity (polar groups) and at least one group with affinity to nonpolar substances (apolar group).

A distinction is made here between nonionic, anionic and cationic emulsifiers.

One characteristic of the hydrophilicity of a given emulsifier is its HLB value, which is given by the following formula: HLB=20=(1−Mₗᵢₚₒₚₕᵢₗ/M), where Mₗᵢₚₒₚₕᵢₗ stands for the molar mass of the lipophilic fraction in the emulsifier and M stands for the molar mass of the total emulsifier.

In general, emulsifiers with an HLB value up to about 8 are considered to be W/O emulsifiers. By contrast, O/W emulsifiers have HLB values of greater than 8 to 15. Substances with HLB values greater than 15 are often referred to as solubility promoters.

According to the invention, it has now been found that when choosing two W/O emulsifiers, which are preferably present in a fraction of more than 1.5% by weight in the preparation and additionally at least one readily spreading oil is present, flowable lotions are obtained which are characterized by care but at the same time non-oily and nongreasy sensorics (see example formulations). Moreover, these formulations are stable over a long period, surprisingly even upon storage at different and/or fluctuating temperatures. As is known, in the production of relatively large product amounts, so-called up-scaling, there are often problems for such formulations. This process, known as “up-scaling”, is known to be particularly sensitive for customary W/O emulsions. For the preparations described within the framework of this invention, the up-scaling process has proven to be surprisingly problem-free, which is based on the emulsifier choice according to the invention.

The cosmetic and/or dermatological preparation according to the invention is a water-in-oil emulsion that is flowable at room temperature and not a multiple emulsion such as W/O/W or O/W/O emulsion. Advantageously, the emulsion according to the invention is not formulated as microemulsion or nanoemulsion.

The invention is preferably a cosmetic or dermatological preparation based on a water-in-oil emulsion which has no (0%) ethoxylated emulsifiers, polyethylene glycols and/or polyethylene glycol derivatives.

Furthermore, the preparations according to the invention advantageously comprise one or more powder raw materials, which are present preferably in a fraction of up to 5% by weight, preferably 0.2 to 2% by weight, based on the total mass of the preparation. Preferred powder raw materials are aluminum starch octenylsuccinate and/or talc.

The cosmetic or dermatological preparations according to the invention can also comprise cosmetic auxiliaries and further active ingredients as are customarily used in such preparations, e.g. preservatives, preservation auxiliaries, bactericides, substances for preventing foaming, dyes and colored pigments, thickeners, moisturizing and/or humectant substances, fats, oils, waxes or other customary constituents of a cosmetic or dermatological formulation such as alcohols, polyols, polymers, foam stabilizers, electrolytes, organic solvents or silicone derivatives, self-tanning agents, buffers, pH regulators, plant extracts, surfactants, propellant gases, powders, sebum-absorbing substances, UV filters, active ingredients such as for example anti-aging, anti-cellulite, anti-acne, anti-rosacea, anti-neurodermatitis, antioxidants, moisturizers, chelating agents, antiperspirants, bleaches and colorants etc., provided the addition does not hinder the required properties as regards freedom from PEG, emulsifier content, required stability and sensorics.

The water content of the preparations according to the invention is advantageously between 40 and 80% by weight, preferably between 50% by weight and 70% by weight, particularly preferably between 55% by weight and 65% by weight, in each case based on the total mass of the preparations.

In the case of restrictions to preferentially mentioned substances, whether lipids with spreadability value according to the invention, the W/O emulsifiers or further preferably mentioned constituents, then their preferred fraction ranges also refer to the individual constituents then selected. The other constituents excluded by the restriction then are no longer added to the listed fraction ranges.

The examples below illustrate the preparations according to the invention. The numerical values refer to the weight fractions with regard to the total mass of the preparation, unless stated otherwise.

### EXAMPLE 1

### 0.5% Polyglyceryl-4 Diisostearate/Polyhydroxystearate/Sebacate

### 1.9% Diisostearoyl Polyglyceryl-3 Dimer Dilinoleate

### 3% Cera Microcristallina

### 10% Isopropyl Myristate

### 10% Dicapylyl Ether

### 1% Talc

### 12% Glycerin

1% Propylene glycol

### 0.1% Hexanediol

### 0.15% Potassium Sorbate

### 0.1% Citric Acid

### 0.2% Sodium Citrate

### 0.4% Perfume

with Aqua ad 100%

### EXAMPLE 2

### 0.8% Polyglyceryl-4 Diisostearate/Polyhydroxystearate/Sebacate

### 1.9% Diisostearoyl Polyglyceryl-3 Dimer Dilinoleate

2.5% Cetyl palmitate

### 0.1% Cera Microcristallina

### 2% C13-16 Isoparaffin

### 2% Cyclomethicone

### 4% Argania Spinosa Kernel Oil

### 0.1% Calendula Oil

### 6.5% Paraffinum Liquidum

### 6.5% Isopropyl Palmitate

### 10% Glycerin

### 2% Tapoca Starch

### 0.15% Potassium Sorbate

### 0.2% Sodium Citrate

### 0.1% Citric Acid

### 0.15% Perfume

with Aqua ad 100%

### EXAMPLE 3

### 1.5% Polyglyceryl-4 Diisostearate/Polyhydroxystearate/Sebacate

### 1.5% Diisostearoyl Polyglyceryl-3 Dimer Dilinoleate

### 2.7% Shea Butter

### 7.5% Isohexadecane

### 5.3% Dibutyl Adipate

### 6.5% Paraffinum Liquidum

### 0.1% Sunflower oil

### 13.75% Glycerin

### 0.5% Ethylhexylglycerin

### 1% Aluminium Starch Octenylsuccinate

### 1% Nylon-12

### 0.15% Potassium Sorbate

### 0.2% Sodium Citrate

### 0.1% Citric Acid

### 0.3% Perfume

with Aqua ad 100%

### EXAMPLE 4

### 0.8% Polyglyceryl-4 Diisostearate/Polyhydroxystearate/Sebacate

### 1.4% Diisostearoyl Polyglyceryl-3 Dimer Dilinoleate

### 1% Cera Microcristallina+Paraffinum Liquidum

### 0.5% Cera Microcristallina

### 2% Paraffinum Liquidum

### 1% Prunus Amygdalus Dulcis Oil

### 0.5% Cetyl Palmitate

9.5% Isopropyl palmitate

### 6% C13-16 Isoparaffin

### 0.5% Aluminium Starch Octenylsuccinate

### 10% Glycerin

### 0.15% Potassium Sorbate

### 0.7% Magnesium Sulfate

### 0.1% Maris Sal

### 0.5% Glyceryl Glucoside

### 0.2% Sodium Citrate

### 0.1% Citric Acid

### 0.35% Perfume

with Aqua ad 100%

### EXAMPLE 5

### 1.4% Polyglyceryl-4 Diisostearate/Polyhydroxystearate/Sebacate

### 0.8% Diisostearoyl Polyglyceryl-3 Dimer Dilinoleate

### 0.2% Cera Microcristallina

### 1% Shea Butter

### 2.5% Cera Microcristallina+Paraffinum Liquidum

### 7.5% Caprylic/Capric Triglyceride

### 12% Dicaprylyl Ether

### 0.1% Olive oil

### 1% Talc

### 0.2% Aluminium Starch Octenylsuccinate

### 12% Glycerin

### 0.1% Caprylyl Glycol

### 0.15% Potassium Sorbate

### 0.1% Citric Acid

### 0.2% Sodium Citrate

### 0.4% Perfume

with Aqua ad 100%

### EXAMPLE 6

### 1.1% Polyglyceryl-4 Diisostearate/Polyhydroxystearate/Sebacate

### 1.2% Diisostearoyl Polyglyceryl-3 Dimer Dilinoleate

### 0.2% Cera Microcristallina

### 1% Shea Butter

### 2.5% Cera Microcristallina+Paraffinum Liquidum

### 7.5% C13-16 Isoparaffin

### 12% Dicaprylyl Ether

### 0.1% Olive oil

### 1% Talc

### 0.2% Aluminium Starch Octenylsuccinate

### 12% Glycerin

### 0.1% Caprylyl Glycol

### 0.15% Potassium Sorbate

### 0.1% Citric Acid

### 0.2% Sodium Citrate

### 0.4% Perfume

with Aqua ad 100%

1.-19. (canceled)

20. A cosmetic and/or dermatological preparation, wherein the preparation is flowable at room temperature and is based on a water-in-oil emulsion comprising at least two W/O emulsifiers and one or more oils having a spreadability value greater than 600 mm²/10 min.

21. The preparation of claim 20, wherein two of the at least two W/O emulsifiers differ in their HLB value by at most 1.

22. The preparation of claim 21, wherein the two W/O emulsifiers differ in their HLB value by at most 0.5.

23. The preparation of claim 20, wherein only two W/O emulsifiers are present.

24. The preparation of claim 20, wherein the at least two W/O emulsifiers comprise diisostearoyl polyglyceryl-3 dimer dilinoleate and polyglyceryl-4 diisostearate/polyhydroxystearate/sebacate.

25. The preparation of claim 20, wherein the one or more oils having a spreadability value greater than 600 mm²/10 min have a viscosity of not more than 10 mPas.

26. The preparation of claim 20, wherein the one or more oils having a spreadability value greater than 600 mm²/10 min are present in a concentration of from 1% to 30% by weight, based on a total weight of the preparation.

27. The preparation of claim 20, wherein the one or more oils having a spreadability value greater than 600 mm²/10 min are present in a concentration of from 30% to 90% by weight, based on a total weight of an oil phase.

28. The preparation of claim 20, wherein the one or more oils having a spreadability value greater than 600 mm²/10 min are selected from one or more of isopropyl palmitate, isopropyl myristate, coco-caprylate/caprate, isopropyl isostearate, cyclomethicone, butylene glycol dicaprylate/dicaprate, dicaprylyl carbonate, isopropyl stearate, ethylhexyl cocoate, dibutyl adipate, isodecyl neopentanoate, isohexadecane, C13-16 isoparaffin, and dicaprylyl ether.

29. The preparation of claim 20, wherein the one or more oils having a spreadability value greater than 600 mm²/10 min consist of one or both of isopropyl palmitate and C13-16 isoparaffin.

30. The preparation of claim 20, wherein the preparation comprises one or more oils having a spreadability value greater than 600 mm²/10 min comprise at least one oil having a spreadability value greater than 1000 mm²/10 min.

31. The preparation of claim 20, wherein the preparation further comprises a total of from 5% to 20% by weight, based on a total weight of the preparation, of at least one skin moisturizer.

32. The preparation of claim 31, wherein the at least one skin moisturizer comprises glycerol.

33. The preparation of claim 20, wherein the preparation further comprises one or more powder raw materials.

34. The preparation of claim 33, wherein the one or more powder raw materials are present in a concentration of up to 5% by weight, based on a total weight of the preparation.

35. The preparation of claim 34, wherein the one or more powder raw materials are present in a concentration of from 0.2% to 2% by weight.

36. The preparation of claim 33, wherein the one or more powder raw materials comprise at least one of aluminum starch octenylsuccinate and talc.

37. The preparation of claim 20, wherein the preparation comprises water in a concentration of from 40% to 80% by weight, based on a total weight of the preparation.

38. The preparation of claim 20, wherein the preparation comprises from 0% to less than 1% by weight based on a total weight of the preparation, of polyethylene glycols and polyethylene glycol derivatives.

39. A method of reducing the viscosity of a cosmetic water-in-oil emulsion, wherein the method comprises incorporating into the emulsion one or more oils having a spreadability value greater than 600 mm²/10 min.
